(19) United States
(12) Patent Application Publication
(10) Pub. No.: US 2004/0175353 A1
(43) Pub. Date: Sep. 9, 2004

(54) Cyanoacrylate compositions for prophylactic or therapeutic treatment of diseases manifesting themselves in and/or damage cutaneous tissue

(76) Inventor: Hans Brinch Lyster, Kastrup (DK)

Correspondence Address: WINSTON & STRAWN, PATENT DEPARTMENT, 1400 L STREET, N.W., WASHINGTON, DC, 20005-3502, US

(21) Appl. No.: 10/619,390

(22) Filed: Jul. 14, 2003

Related U.S. Application Data
(63) Continuation of application No. PCT/DK02/00034, filed on Jan. 16, 2002.

(30) Foreign Application Priority Data
Jan. 16, 2001 (DK) PA 2001 00067
Jan. 16, 2001 (DK) PA 2001 00068
Feb. 16, 2001 (DK) PA 2001-00264

(51) Int. Cl. A61K 31/785

(52) U.S. Cl. 424/78.35

(57) ABSTRACT

A novel method for the prophylactic or therapeutic treatment of skin disorders and diseases by the application of a medicament that includes at least one cyanoacrylate compound. The medicament offers an advantageously novel alternative to hitherto known medicaments for the treatment of exanthemas, psoriasis exanthemas, and exanthemas in connection with fungal infections, as well as virus infections, and revision of infected and non-infected wounds.

### CROSS-REFERENCE TO RELATED APPLICATION

This is a continuation of International Application PCT/DK02/00034, filed Jan. 16, 2002, the entire content of which is expressly incorporated herein by reference thereto.

### FIELD OF INVENTION

The invention relates to a method for prophylactic or therapeutic treatment of skin disorders or diseases which manifest themselves in or damage cutaneous tissue, wherein the method includes application of at least one cyanoacrylate. The invention also relates to the revision of wounds without the need for surgery, and the removal of exanthema plaque, condyloma, and other warts.

### BACKGROUND OF THE INVENTION

Cyanoacrylate adhesives and cyanoacrylate-based compositions are known for treating or preventing certain skin problems. For example, WO 95/00153, describes covering a selected skin area that is exposed to friction or irritation with a protective cyanoacrylate polymer coating to prevent friction contact with the selected skin area, thereby preventing friction contact with the selected skin areas and reducing the risk of the formation of deep and slow-healing lesions. Likewise, U.S. Pat. No. 5,306,490 also discloses coating a skin area with a cyanoacrylate polymer coating for the prevention of blister formation.

Additionally, WO 97/47310 and WO 98/03152 both describe a glyceryl poly(meth)acrylate gel for treating or preventing different skin disorders. For example, in the cases of candida infections, Herpes Simplex 1 and 2 infections, sunburns and/or irritation of oral mucous membrane, the water absorbing and osmotic properties of the gel contributes to the dehydration of the micro-organisms present and which therefore will no longer be able to survive.

Other examples of using cyanoacrylates for skin problems include using the a cyanoacrylate-based adhesive to glue together edges of skin which cannot be sutured, or only can be sutured with difficulty, as described in WO 93/25196. Such application largely takes place in hospital emergencies. Another adhesive cyanoacrylate-based composition is known from WO 01/012243.

Moreover, cyanoacrylate-based compositions are also known in the treatment of corneal lesions, as described in “Tissue adhesive arrest stromal melting in the human cornea”, J. A. Fogle, M.D., Kenneth R. Kennyon, M.D., and C. Stephen Foster, M.D., Am. J. Ophthalmol. 1980, vol. 89, no. 6, pp. 795-802. As described therein, an isobuthyl-2-cyanoacrylate was applied together with bandage lens following surgical removal of sick tissue and supplemented with subsequent medical post-surgical treatment. In this case, the cyanoacrylate polymer coating serves as plaster under which the wound is allowed to heal during a long period.

Also known is the cosmetic removal of impurities and dead cells from the skin surface, and especially from sebaceous follicles in the skin by applying cyanoacrylate adhesive over the desired area to be cleaned is described in U.S. Pat. No. 4,752,472. The impurities attach to the polymer coating and then can be pulled off after a short time, thereby cosmetically removing such impurities and dead cells from the skin surface.

It is also known that viral infections of the skin and the mucous membrane often result in long and recurring nuisances in the form of itching, prickling, vesiculation ulcerations, and the like. Some types of viruses can even cause inconvenient and disfiguring excrescences such as warts and condylomas. An example of such a viral infection is Herpes.

Herpes around the mouth is due to the virus of the Herpes Simplex I (HSV-I) type which also can cause infections on the nose, the cheeks and the chin together with the oral cavity. Herpes in the genital region is due to the virus of the Herpes Simplex II (HSV-II) type. The former of the two types of herpes is known as Herpes labialis and the latter as Herpes genitalis.

A very common form of Herpes Simplex I causes blisters, vesicles or wounds on the skin and mucous membrane at the mouth. These wounds are often very large and the crust confluent and downright disfiguring the patient's appearance. Moreover, the Herpes virus is very infectious, and the patient can easily spread the infection to large skin or mucous membrane areas by mere touch.

Yet another form of Herpes Simplex I causes herpetic gingivitis which is identified as small, very painful lesions on the inside of the mouth and even in the throat.

In contrast to the Herpes Simplex I, Herpes Simplex II is only found below the belt. Women typically get symptoms in vulva, vagina and cervix whereas men typically get symptoms on penis, especially on the prepuce or on corpus but also in the surrounding genital areas. Direct inoculation of virus takes place upon contact with infected secretions or infected mucous membranes.

In addition to the Herpes viruses, another virus that especially attacks the skin is the papilloma virus type which among other things causes condylomas. Condylomas are small papillomas with a central core of coherent tissue covered by epithelial tissue. Outbreaks often occur on the mucous membrane in the extragenital regions or in the perianal region.

An often used commercially available medicament for the therapeutic treatment of Herpes Simplex is aciclovir (for example Zovirax™, Glaxo-Wellcome, Inc.), which is a nucleoside analogue acting by selectively inhibiting the synthesis of viral DNA. Acyclovir is phosphorylated by the viral thymidine kinase and can, as triphosphataciclovir, inhibit DNA polymerase and thereby the formation of viral DNA (Informed Drug Guide, Informed-Verlags AG, Germany 1996). Unfortunately, the prophylactic treatment of Herpes with oral aciclovir only has a minimal effect on recurring infections but is the preferred treatment for genital herpes infection.

Presently, a number of alternative chemical formulations exists for treatment of different types of herpes infections. Examples of such formulations are famciclovir (Famvir™, SmithKline Beecham) for treating especially herpes zoster, valaciclovir.HCl (Valtrex™, Glaxo-Wellcome, Inc.) for treating especially Herpes Simplex, BV-araU (Sorivudin™, Bristol Myers Squibb) for treating especially herpes zoster, and Foscarnet (Foscarvir™, Astra Pharmaceuticals) for treating several different types of herpes.

Disadvantageously, these antiviral medicaments are long-term treatments, and are expensive for patient's to use and are therefore only used at such a late phase in the course of the disease. Additionally, these medicaments cannot effectively arrest the further development of the disease.

Another disadvantage of the presently known treatments is that use of aciclovir ointment on Herpes around the mouth, the patient is left with a greasy, visible ointment deposition on the infected area. Application has to be repeated frequently, and the appearance of the patient is disfigured to such an extent by the ointment patch that the patient in many cases is hindered in his work where a presentable appearance often is required.

Other types of skin diseases or disorders such as condylomas can be treated locally with cryotherapy and other forms of physical action. However, these treatments are often unpleasant and accompanied by pain. Oral or parenteral treatment with e.g. interferon is also known but is often accompanied by complications in form of fever, myalgia and headaches, and the costs of these forms of treatment are in themselves prohibitive.

Furthermore, other forms of exanthema, such as eczema, psoriasis exanthema or fungal infection in the skin or in the mucous membrane can be caused by a number of different known or unknown diseases or actions. Examples of such exanthema include atopic eczemas known as e.g. infantile eczema, asthma eczema, atopic dermatitis or Prurigo Besnier, seborrhoeic exanthema, discoid exanthema, allergic contact dermatitis or irritation contact dermatitis.

The presently known treatment for exanthemas aims at removing the substances or conditions that provoke the exanthema and to relieve the pruritus and the infection condition. The treatment of the exanthema itself often takes place by means of cream containing adrenal cortical hormone. Examples of such creams are Dermil®, from Nettopharma, Hydrocortisone “DAK” cream, Mildison® from Pharmaco Ltd., Uniderm® from Schering-Plough A/S, Corticoderm® (from Pharmacia & Upjohn, Locoid® from Yamanouchi Pharma A/S, Diproderm® from Scheering-Plough A/S, Elocon® from Scheering-Plough A/S, Ibaril® from Hoechst Marion Roussel A/S, Synalar®from Bioglan Pharma Plc., Diprolen® from Scheering-Plough A/S, and Emovat® from Glaxo Wellcome A/S.

However, the use of adrenal cortical hormone often prove only to have effective effect upon use for a longer period of time which often involve a number of adverse effects. The skin becomes dry, thin and brittle which can be seen as small haemorrhages in the plexus of the blood vessels of the skin and the resistance to micro-organisms is reduced. Some people develop allergy to adrenal cortical hormone and their eczema is even aggravated. Therefore, it is not expedient or desirable to treat with strong adrenal cortical hormones, and especially treatment of children, expectant and breast-feeding mothers should be avoided completely.

Another form of exanthema is connected with psoriasis which is divided into two main groups psoriasis vulgaris and psoriasis pustulosa. Psoriasis is a chronic, recurring disease which in some cases can be socially disabling and in rare cases even potentially lethal. Psoriasis vulgaris is the most frequent form. It most often start with small, red spots or plaques. The areas will gradually grow and start scaling. The top scales fall off in relatively large amounts but the bottom scale layers are firmly fixed. If removed, Auspitz sign will show, that is small haemorrhages in the skin under the scales. Psoriasis vulgaris often exists symmetrically over the entire body and affects especially elbows, knees, groin, arms, legs, scalp, and nails.

Nail psoriasis manifests itself as small depressions in the nails that can resemble the depressions in a thimble and the nails can be so severely affected that they are thickened, crumble and fall off.

Inverse psoriasis is found in skin folds, such as armpits, under the breasts, and in skin folds on the stomach around the groin and buttocks where red, irritating plaques are often infected with Candida albicans.

Guttate psoriasis is a psoriasis variant which is primarily provoked acutely in children and youth after a streptococcal infection of the throat. The exanthema manifests itself as many, drop-like, scaling patches over the entire body.

Psoriasis on the scalp resembles seborrhoeic dermatitis and occasionally the two skin diseases occur at the same time.

Pustular psoriasis is a psoriasis variant in which the sterile inflammation reaction is so violent that besides the usual lesions, pustules are also formed.

Today, the treatment which varies depending on the patient's age and the type of the character of the disease comprises different local treatments and whole-body treatments with creams, ointments and liniments. This medical treatment is often supplemented with light treatments, tar baths, climotherapy and other special treatments. Examples of tar-containing medicine which dissolve plaques are Basotar ® cream from Galderma Svenska og Inotyol® ointment from A/S GEA.

The medicament Diavonex ® from Løvens Kemiske Fabrik inhibits the growth of sick skin cells in a psoriasis-affected skin and scalp and is used for local treatment. Daivonex® is found as cream, ointment and liniment and is based on a synthetically manufactured vitamin D-derivate which is working by stimulating the formation of normal skin cells.

Examples of adrenal cortical hormone-containing medicaments which are used for supplementary local treatment are Emovat® from Glaxo Wellcome A/S, Dermil® from Nettopharma, Mildison® from Pharmaco Ltd, Uniderm® from Scheering-Plough A/S, Corticoderm® from Pharmacia & Upjohn and Hydrocortisone “DAK” cream from DAK.

A few immuno-suppressing agents for systemic treatment of a very severe psoriasis also exist. An example of such an immuno-suppressing agent is Emthexate® from Nettopharma. The agents act by killing both healthy and sick cells. The agent is among other things, acting by inhibiting the conversion of folic acid and thereby inhibiting, among other things, the cell construction of DNA which therefore becomes defective which causes the cell to perishes. Even if the medicament is only used in small doses for psoriasis treatment, side effects are often found such as nausea, diarrhoea, leucopenia, hair loss and affections of mouth and intestinal mucosa in form of wounds and coatings in the mouth.

Another well-known exanthema is caused by infections of fungi or yeast. Examples are e.g. Trichophyton, Epidermophyton, Candida, Torulopsis, Cryptococcus, Pityrosporon or Trochosporon, which quickly can manifest themselves as exanthema in human dermal or human mucous tissue.

Fungi subsist on dead skin cells and typically grow as articulated threads in the skin. The infections are very infectious, they spread in moist and warm environments and are infectious via both direct and indirect contact. Cutaneous fungus can be caused by many different types of fungi and have similarly varied symptoms. However, a common symptom is often an itching or stinging reddish exanthema with small blisters in connection with the infection.

Common to the above skin disorders and diseases is that they all manifest themselves as an exanthema, which is troublesome to the patient, and itches, forms blisters or scales and is cosmetically annoying. The exanthemas usually require treatment that are difficult to treat and the treatments are often long-term. In addition, the present medicaments for treating these exanthemas are known to involve more or less serious side effects.

In addition to the skin disorders mentioned above, another skin disorder is a wound. A wound is defined by the expert as an open lesion of exterior and interior surface of an organism, for example a lesion of the skin on a human.

Types of wounds in humans can be classified as wounds with tissue loss on the surface of the body, and divided primarily into acute wounds and chronic wounds. The acute wounds heal up normally and often without complications, where the chronic wounds which are caused by an underlying disease have a very slow healing which often stop completely. Especially large, acute and chronic wounds are very difficult to keep clean, and such wounds can easily be infected with microorganisms, such as bacteria, fungi and vira, which multiply and invade either the deeper part of the wound, the wound edges and possibly the wound surroundings, which inhibit the healing and necessitate revision of the wound. Also smaller chronic wounds in patient with reduced or poor immune defence heal slowly and therefore often get infected.

The acute wounds, vulvus, are produced after exterior trauma and comprise surgical wounds and infections-conditioned wounds, for example after operation, traumatic wounds, such as stab wounds, abrasions, burns, etching, and frost-bites.

The chronic wounds, ulcus, are caused by a disease process and comprise for example pressure sores, bedsores, venous- or arterial-conditioned wounds or combination wounds of these wounds, wounds caused by diabetes mellitus, infected wounds or fistulas.

A wound is further divided into the types black, yellow and red wounds, and it is this division that is applied in relation to determination of local treatment.

The black wound is the most serious and is recognised by its black necroses which are found in and/or is covering the wound. These result in a delayed healing through inhibition of epithelization, ingrowth of fibroblasts to connective tissue healing and an increased collagenosis. Finally, an increased bacterial growth can occur during a necrosis resulting in infection. Black necroses at arterial and diabetic wounds are especially seen in case of wounds related to vessel changes. The treatment is most often removal of the necrosis by surgical wound revision. As it is a painful process to revise a wound, the physician will in many cases defer the revision until the necrosis will get loosen at the edges by itself or await a spontaneous exfoliation. Alternatively, the wound revision must often take place using a form of anaesthesia.

The yellow wound consists of changed fatty tissue, connective tissue residues and fibrin precipitation. This type of wound, which is most frequently seen in arteriosclerotic and diabetic wounds but rarely in venous wounds, are generally less important to the healing than the black wound. The treatment can comprise wound cleaning, possibly with the addition of a wound revision when infections supervene.

The red wound primarily consists of granulation tissue, the appearance of which has named the wound. After correct treatment, black and yellow wounds will reach this phase in which the wound usually heals quickly, and the wound revision aspires to promote formation of the read wound.

Revision of a wound comprises removal, most often surgical, of the crust and unhealthy surrounding tissue, such as necrotic tissue. The wound revision is often supplemented with a prophylactic and/or therapeutic treatment, such as with antibiotics or antifungal drug.

The currently methods for treating the multitude of skin disorders and diseases outlined above are expensive, long-term, and often do not arrest or inhibit further development of the skin disorder. Moreover, often there are unwanted side effects to the treatment. In the case of wound revisions, the treatment is often surgical. Thus, there remains a need for an easy, inexpensive, effective method for treating these skin disorders and diseases, without or only with mild and few side effects.

### SUMMARY OF INVENTION

The novel and unique features according to the invention are accomplished treating skin disorders or diseases, wounds, warts, or condylomas with medicament including a cyanoacrylate having the general monomer formula I,

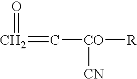

wherein R preferably is chosen from the group of alkyls or alkenyls having 1 to 10 carbon atoms, cycloalkyls having 5 to 10 carbon atoms, phenyl, 2-ethoxyethyl, 3-methoxybutyl, arenes or alkyl-substituted arenes or a substituent having the formula II:

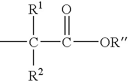

wherein

R¹ and R² are chosen independently of each other from the group consisting of hydrogen, methyl, ethyl, propyl or butyl, and

R″ is chosen from the group consisting of alkyls or alkenyls having 1 to 10 carbon atoms, cycloalkyls having 3 to 10 carbon atoms, or R″ is chosen from the group consisting of phenyl, benzyl, methylbenzyl, phenylethyl or halogen-substituted or alkyl-substituted compounds of these.

In the case of treating exanthema, the method includes applying cyanoacrylate directly on an infected area of the skin tissue, the cyanoacrylate polymerizes and forms a polymer coating on the selected area of tissue, which inhibits development of the exanthema.

The method includes treating various types of exanthema including but not limited to eczema, psoriasis, fungal, and the like.

In another embodiment of the invention, a viral infection is treated with the application of a medicament including a combination of cyanoacrylate monomers. Such combination of cyanoacrylate monomers may have different R groups.

In yet another embodiment of the invention, the method includes revising a wound without the need for surgery. The cyanoacrylate is applied to the wound, which preferably has a crust thereon, the cyanoacrylate polymerizes into the crust of the wound, the wound crust is pulled off the skin surface by pulling the cyanoacrylate polymer off the skin surface, which is adhered to the crust.

The invention also includes a method for maintaining in close proximity separated tissue edges resulting from a wound. In this aspect of the invention, cyanoacrylate is applied to the separated edges of the wound such that flaps are formed by an excess of cyanoacrylate. The flaps are grasped, and the edges of the tissue are placed in close proximity to facilitate closure procedures such as suturing.

Some of the many advantages of the present invention include providing a medicament comprising cyanoacrylate to prevent, hinder and treat outbreaks of viral infections, or skin exanthema in human or animal cutaneous tissue or in the mucous membrane tissue, quickly, less expensively and without cosmetic nuisances to the patient. Advantageously, the medicament also can be used by children, as well as expectant and breast-feeding mothers.

Other advantages of the invention include removing plaque associated with exanthema, and other skin disorders such as condolyma and warts more quickly, easily and less expensively and without painful nuisances to the patient. The method of the present invention completely or partly eliminates after-treatment of the skin disorder or disease with antibiotics, penicillin and/or fungicidal drugs, steroids, and the like.

### DETAILED DESCRIPTION OF THE PREFERRED EMBODIMENTS

The inventor of the present invention has surprisingly found that application of a liquid cyanoacrylate adhesive on a viral infected area of skin and/or mucous membrane inhibits or prevents the clinical manifestation of the viral infection.

The medicament of the invention is preferably used for prophylactic or therapeutic treatment of virus types such as Herpes Simplex 1, Herpes Simplex 2, herpes zoster or papilloma virus.

The capillary effect from the liquid cyanoacrylate means that the liquid can penetrate into all the cavities of the skin or mucous membrane, and thus place a completely tight-fitting coat over the infected body area.

The inventor who is medical specialist in general medicine has surprisingly found that in the cases of a herpes infection having reached a stage of development that symptomatically is characterised by liquid-filled blisters in an area of skin and/or mucous membrane, application of a small amount of cyanoacrylate on such a blister arrests the development of the infection. The tissue fluid in the blister is utilised by the cyanoacrylate to initiate polymerization, and due to the formed polymer coat, possible further virus-containing liquid in the blister can be encapsulated so that spreading to larger areas of skin or mucous membranes can easily be prevented. The contact with the tissue fluid causes rapid polymerization even when the cyanoacrylate is merely contacted with water and also results in the formation of a far stronger and harder polymer coat.

In cases of a ruptured blister and the commencement of ulceration, the crust is easily removed by applying a small covering amount of cyanoacrylate across the crust, the humidity content of which causes the polymerization to pass off quickly.

After polymerization, the cyanoacrylate is securely stuck in the crust which subsequently can be pulled off the infected area easily and painlessly. In this way, ugly and scaring crusts can quickly be removed, and the risk of bacterial contamination of the virus-infected area is easily prevented by applying a new cyanoacrylate coat which in addition advantageously inhibits or stops the virus from developing continuously. The symptoms of the virus infection have completely disappeared within about 1-4 days.

The medicament may also be used for the treatment of condylomas and warts. The condylomas or warts are encapsulated in the cyanoacrylate coat, and the entire or at least parts of the condylomas or warts can be removed in a manner similar to the crusts mentioned above. By repeating the treatment, condylomas or warts can disappear completely.

The outbreak of viral infections in the skin and mucous membrane can easily be prevented from manifesting themselves visibly, with the use of cyanoacrylate, and such use is a novel and inexpensive alternative to known antiviral medicaments. Thus, the medicament of the present invention allows a patient to treat himself easily, inexpensively and when needed.

The inventor of the present invention has also, based on his own experiences, found that application of a liquid cyanoacrylate adhesive on an area of skin and/or mucous membrane with exanthema makes it possible to remove exanthema completely from the area of skin or mucous membrane. A skin area with e.g. Prurigo Besnier is left completely smooth and silky for a longer period of time after merely a single treatment. The medicament is preferably used for the prophylactic or therapeutic treatment of exanthemaes such as atopic exanthema, seborrhoeic exanthema, discoid exanthema, allergic contact dermatitis or irritation contact dermatitis. When the cyanoacrylate polymer is applied on the infected area in a thin layer, the cyanoacrylate polymer polymerizes quickly to a coherent polymer coat, in which occurrences on the skin or mucous membrane of skin-irritating elements such as pollen, cream or soap residues or similar artificially supplied foreign bodies, unhealthy tissue, dead skin cells, vira, bacteria and yeast or fungi are absorbed, and it has surprisingly turned out that by means of the medicaments according to the invention, it is possible to clean the skin so effectively that minor exanthema is eliminated completely and that subsequent medical treatment is not necessary.

In the case of severe exanthemaes, several treatments with the medicament according to the invention can be advantageous. The treatment can possibly be done periodically with or without interruptions with supplementary treatment with e.g. moistening or pH-adjusting creams, fungicides, antibiotics or the like, but it goes without saying that the sooner an exanthema is treated with the medicaments according to the invention, the easier and more quickly will it be to limit the extent and degree of the exanthema and thereby of a possible supplementary treatment.

The medicaments according to the invention is especially advantageously applicable when the skin exanthema is a psoriasis exanthema, where especially plaque appearances easily can be removed by one or several treatments.

The capillary effect from the liquid cyanoacrylate means that the liquid can penetrate into all the cavities of the skin, mucous membrane and plaque, and thus place a completely tight-fitting coat surrounding the plaque which subsequently merely can be removed together with the polymer coat.

In case of treatment of e.g. nail psoriasis with cyanoacrylate compounds according to the present invention, the nail can be filed after application of cyanoacrylate and thereby obtain a cosmetically acceptable state as well as reinforcing a normal nail by applying cyanoacrylate.

A further advantageous use is seen when the skin exanthema is caused by a fungal infection. Normal fungal infections in the skin can occur after infection with fungi of the strains Trichophyton, Epidermophyton, Candida, Torulopsis, Cryptococcus, Pityrosporon or Trochosporon, and especially the species Candida albicans or Pityriasis versicolor cause many fungal attack. Initial studies indicate that Candida possibly is devitalised, which is why the medicament according to the invention provides an advantageous, quick-acting alternative to existing forms of treatment.

As mentioned above, the cyanoacrylate is securely stuck in loose components after polymerization, and the loose components of the exanthema can therefore be pulled off the infected, now completely cleaned, attacked tissue area easily and painlessly, and for example a psoriasis plaque can be pulled completely off after one or several treatments and give the psoriatic a renewed quality of life.

The risk of bacterial contamination of the cleaned area can be prevented if necessary by either applying a new cyanoacrylate polymer coat or by prophylactic treatment with relevant supplementary pharmaceuticals.

The use of cyanoacrylate compounds for prophylactic and therapeutic treatment of a large number of such skin exanthemaes therefore constitutes a novel, inexpensive and effective supplement to forms of treatment known today.

The cyanoacrylate compounds and compositions containing different cyanoacrylate compounds have not previously formed part of medicaments for revision of acute or chronic wounds. The chronic wounds especially require treatment when the wounds are found as black, necrotic wounds. Often acute wounds develop into chronic wounds which necessitates wound revision. In case of e.g. burns, the additional advantage is obtained in that application of a cyanoacrylate polymer coat contributes to prevent the loss of often large amounts of fluid.

The liquid cyanoacrylate compound has the advantage that it, when spread over a crust, can penetrate into and be distributed into the cavities of the crust. Upon contact with the tissue fluid, the polymerization of the cyanoacrylate monomers begins which gradually creates a polymer network and a coat that adhere to the crust effectively, unbreakably and securely.

By means of a cyanoacrylate polymer coat which will be formed after complete polymerization which will be completely completed already after 1 minute for most cyanoacrylate compounds and combinations of cyanoacrylate compounds, even a relatively thick crust can be pulled off easily and quickly, and the exposed, open lesion can be cleaned out and after-treated further according to individual needs and discretion. The reaction time depends on the applied amount of cyanoacrylate and the surface of the wound, and can preferably be as short as under 10-15 seconds.

The wound can easily be rinsed and cleaned out by means of conventional cleansing methods, and possible wound debris, contaminates or other impurities can be removed by means of a supplementary treatment with the cyanoacrylate medicament and subsequent pulling off of these components by means of the formed polymer coat.

The polymer can only adhere very loosely to healthy tissue, and the cyanoacrylate medicament can preferably be placed across the wound in such a way that the hardened polymer coat is extending beyond the limitations of the crust or wound in over the healthy skin so that a detachable edge region can serve as gripping region during the pulling off of the crust or components desired removed. A residue of the polymer coat can possibly be left as an especially advantageous protection of the wound edge.

As medicaments, as mentioned earlier, only partly adhere to healthy tissue, wounds contaminated with various foreign bodies such as e.g. glass splinters, remains of suture material or gunpowder burns, can be cleaned out easily and painlessly.

A péans damages the tissue around which it is clamped and in replacement of such a péans, a polymer coat formed by the medicament according to the invention can be used to grip two wound edges and retain them while they are drawn together so that they can be sutured.

The medicament can e.g. advantageously be used in connection with removal of wounds covering sutures which are to be removed from a surgical incision. Also undesirable hair growth in a region around a wound, or hair desired removed prior to the making of a surgical incision can easily be removed. The polymer coat will settle around the foreign bodies such as the glass splinters or the undesirable hairs respectively and capture and retain these for subsequent removal when the polymer coat is pulled off the wound.

When using the medicament according to the invention for revision of wounds, the patient can advantageously avoid a surgical operation which is time-consuming to both patient and surgeon as removal of crusts by means of cyanoacrylate compounds and compositions of these can take place without use of surgery. In by far the most cases, the preceding local anesthesia which often is necessary in case of surgical wound revision can advantageously be spared when the present medicament is used for wound revision.

After pulling off of the crust, a possible antibiotic, fungicidal drug, or drug for treating eczema can be applied to the revised wound and subsequently a new cyanoacrylate polymer coat can possibly be applied to cover the treated wound in replacement of a conventional bandage or conventional plaster.

It is especially preferred to use a cyanoacrylate polymer coat for protecting a revised, possibly further treated wound on sites where it is difficult to fasten a bandage or a plaster, such as e.g. around articulations and under mammae, or in situations where it will be an advantage that the bandage can follow the movements of the body unobstructedly without being removed from the wound.

The cyanoacrylate compounds do not adhere to vital skin/tissue, and the difference between the adhesion ability to vital skin/tissue and the attack of skin/tissue forms a joint basis of the treatment of the above diseases.

A large number of the above-mentioned different substituents known per se R, R¹, R² and R″ can be used, and they can all form part of a cyanoacrylate polymer for producing a medicament according to the present invention.

The medicament can be produced by using a single cyanoacrylate compound or the medicament can comprise a combination of cyanoacrylates in which optionally R, R¹, R² and R″ can be identical or different.

Preferably, a simple, inexpensive ethyl cyanoacrylate compound is used for producing the medicaments according to the invention.

Such an inexpensive, liquid ethyl cyanoacrylate compound is for example commercially available from Loctite European Group, Arabellastrasse 17, D-81925 Munich, Germany, under the name LOCTITE 411. Ethyl cyanoacrylate of this kind is sold for adhering e.g. plastic parts for medical equipment. Another commercially available and applicable cyanoacrylate compound is sold under the commercial name LOCTITE SUPER ATTAK.

Cyanoacrylate compounds have previously been used for gluing wounds together, and there is therefore no health risk in using cyanoacrylate compounds for this new application.

Advantageously, the medicament can furthermore comprise one or more additives. A stabilizer which can prevent the medicament from polymerizing spontaneously during storage can extend the life of the medicament. Stabilizers having a pH equal to or under 7 and which can be neutralized upon contact with moisture is especially preferred.

In some cases, it can also be an advantage to add an agent such as a C1-C10 alkane, ketone or alcohol which can accelerate the polymerization reaction so that the formation of the polymer coat can take place within merely a few seconds.

LOCTITE 41 contains both a stabilizer and an agent for accelerating the polymerization reaction.

The medicament containing the cyanoacrylate compound can either be liquid or in form of a gel. The gelling properties can be provided by e.g. letting the monomers polymerise partly to e.g. di- or trimers in order to thereby make the medicament more viscous and thereby more suitable to settle closely and locally over the area requiring treatment which may have a variable topography and morphology without problems.

The medicament is an attractive alternative to existing medicaments without known side effects. The medicament is simple to use and the treatment often short and easily done by the person himself.

To this should be added that the medicament is an attractive alternative to existing medicaments as it is simple to use, the treatment is short, and the polymer coat almost invisible and therefore does not disfigure the patient's appearance.

For many of the applications, the medicament can advantageously be added an inert colorant so that it is possible to easily locate the limits of the polymer coat when e.g. a crust is to be pulled off.

For other purposes when e.g. a patient is using the cyanoacrylate polymer coat on a visible location through a longer period of time, it will be an advantage if the polymer coat is almost invisible and therefore not disfiguring the patient's appearance. The coat can possibly be covered with powder or rouge.

### EXAMPLES

The present invention will be described in details with reference to the subsequent examples.

All patients participated after informing guidance of their own free will in the tests mentioned below. The treatment with the medicament according to the invention was done by the inventor who is a qualified medical specialist in general medicine and has practiced as medical specialist and district medical head doctor in Osby, Sweden.

Examples 1-3 are initial studies describing three patient groups at different stages of development of HSV-I infection around the mouth which has been treated with ethyl cyanoacrylate (LOCTITE 411, Henkel Loctite Adhesive Ltd.). The infected areas were applied about ⅓ drop of liquid ethyl cyanoacrylate by means of a toothpick and the polymerization was allowed to pass off.

Examples 4-6 are initial studies describing three patient groups having different types of exanthema which has been treated with ethyl cyanoacrylate (LOCTITE 411, Henkel Loctite Adhesive Ltd.). The infected areas were applied liquid ethyl cyanoacrylate and the polymerization was allowed to pass off.

Examples 7-12 are comparative examples of patients treated with ethyl cyanoacrylate (Loctite Super Attak®, Henkel Loctite Adhesive Ltd.) and conventional treatment respectively.

### Example 1

Patient group 1 consisted of three patients without visible symptoms. The group was treated prophylactically as mentioned above under latent HSV-I infection, pronounced prickling and itching in the mucous membrane at the lip being the cause of the treatment.

The ethyl cyanoacrylate coat was removed after treatment overnight and no patients in Patient group 1 developed visible blisters or wounds.

### Example 2

Patient group 2 consisted of 2 patients with visible blisters in the corner of the mouth due to infection with HSV-I. The group was treated initially for 3 hours, after which the ethyl cyanoacrylate coat with content of virus and tissue was removed. The treatment was repeated 2 times for 24 hours, after which the coat was removed. There were no visible traces after the outbreak and both patients were free of symptoms without scar formation.

### Example 3

Patient group 3 consisted of 4 patients all having large suppurating crusts in a large area around the mouth. The group were initially treated by applying liquid ethyl cyanoacrylate which was allowed to polymerise. The polymer coat with adhering crust was removed immediately after polymerization, and the treatment repeated 4 times for 24 hours. After this, three patients had no symptoms or scar formation. The fourth patient was treated for further 24 hours and subsequently had no symptoms or scar formation.

### Example 4

Patient group 1 consisted of three patients having psoriasis plaque on the elbow. After only one application of cyanoacrylate, visible plaque was almost completely removed. Further tests to examine the skin under the plaque were performed at present to form basis for optimization of the choice of cyanoacrylate compound, its viscosity grade and hardening rate and the thickness of the applied layer.

### Example 5

Patient group 2 consisted of two patients having classic outbreaks of Prurigo Besnier. Cyanoacrylate was applied to an area of about 1 times 5 cm. After polymerization, hardening and standing for about 10 min., the cyanoacrylate polymer coat was removed. The subcutis appeared visible in a few areas on about ½×½ mm where the eczema had penetrated the epidermis, and the skin moreover appeared completely free of eczema. Healing of said microscopic areas was completely without problems and after 14 days, there were no suggestion of eczema in the treated area. The tests showed convincing effect of the medicament.

### Example 6

Patient group 3 consisted of two patients treated for Candida infection. Three Candida-attacked areas on each patient were in this comparative test treated with three different combinations of medicaments. Brentan® which is a known effective drug from Janssen-Cilag A/S for treating fungal infection, is working by destroying the cell membrane of the fungus whereby the membrane perishes. A first area was treated with Brentan® alone, a second area was treated with Brentan® in combination with cyanoacrylate, and a third area was treated with cyanoacrylate alone. The three differently treated areas received all 5 treatments. After the treatments, the third area which only was treated with cyanoacrylate was almost normal skin without visible Candida-attack. The second area which was treated with Brentan® alone was unchanged and required further treatment. The first area which was treated with both Brentan® and cyanoacrylate appeared with a slight reddening but had no visible signs of Candida-attack.

### Example 7

### Comparative Study of Treatment of Patients with Herpes Labialis

8 patients from Group A (patient H1-H8) consulted a physician after repeated herpes exanthema. All 8 had used antiviral ointments without useful effect. 6 patients (patient H9-H14) from Group B all had herpes exanthema for the first time.

Cyanoacrylate application was done over the attacked tissue area with Loctite Super Attak® for successive days as stated in Table I below. The polymer coat was allowed to polymerise upon contact with the tissue fluid and left in situ. The polymer coat was removed before new application.

The results of the comparison between the traditional antiviral treatment form and the treatment form according to the invention are given in Table I below. The tests show that by means of the medicament according to the invention, it is possible to arrest and treat an outbreak of Herpes labialis exanthema far quicker and more effective than hitherto known by means of a simple, easy useable medicament without known side effects for a period depending on the character and extent of the outbreak. The patient can treat himself after a short instruction.

TABLE I

| Patient No. | Group | Treatment form | Dose and duration of treatment | Treatment result |
|---|---|---|---|---|
| H1 | A | acyclovir tablets | 5 days, 200 mg × 5 | Still some |
| | | | | exanthema |
| H1¹⁾ | A | acyclovir tablets | 10 days, 200 mg × 5 | Symptom free |
| H1²⁾ | A | cyanoacrylate application | 5 days (3 times a day) | Symptom free |
| H2-H4 | A | acyclovir tablets | 5 days, 200 mg × 5 | No effect |
| H2 | A | cyanoacrylate application | 4 days (3 times a day) | Symptom free |
| H3 | A | cyanoacrylate application | 8 days (3 times a day) | Symptom free |
| H4³⁾ | A | cyanoacrylate application | 4 days (3 times a day) | Visible effect |
| H5 | A | acyclovir ointment | 14 days as required | No effect |
| H5⁴⁾ | A | cyanoacrylate application | 4 days (3 times a day) | Symptom free |
| H6 | A | acyclovir ointment | 14 days as required | No effect |
| H6⁴⁾ | A | cyanoacrylate application | 8 days (3 times a day) | Symptom free |
| H7⁵⁾ | A | cyanoacrylate application | 5 days (3 times a day) | Symptom free |
| H8⁵⁾ | A | cyanoacrylate application | 7 days (3 times a day) | Symptom free |
| H9^{5,6)} | B | cyanoacrylate application | 1 day (3 times a day) | Symptom free |
| H10^{5,6)} | B | cyanoacrylate application | 2 days (3 times a day) | Symptom free |
| H11^{5,6)} | B | cyanoacrylate application | 2 days (3 times a day) | Symptom free |
| H12 | B | antiviral ointment | 14 days as required | No effect |
| H12^{4,6)} | B | cyanoacrylate application | 2 days (3 times a day) | Symptom free |
| H13^{5,7)} | B | cyanoacrylate application | 4 days (1 time a day) | Symptom free |
| H14^{5,7)} | B | cyanoacrylate application | 2 days (1 time a day) | Symptom free |

¹⁾In consequence of the fact that patient 1 still had a little exanthema, the conventional treatment with aciclovir tablets continued for a further 5 days.
²⁾After 14 days without antiviral treatment, Patient 1 had a recurrence with renewed severe outbreak of Herpes labialis which was treated according to the invention.
³⁾Patient 4 did not show up after 4 days of treatment.
⁴⁾Cyanoacrylate painting took place immediately after 14 days of treatment with antiviral ointment.
⁵⁾No previous antiviral treatment
⁶⁾Self-treatment at home
⁷⁾Treatment under medical supervision (the inventor)

### Example 8

Comparative study of skin removal from feet with cyanoacrylate treatment and on the other foot with conventional dermatological treatment in form of filing off and bandaging. When not obtaining success by conventional treatment, cyanoacrylate treatment continued. The results are given in Table II below.

TABLE II

| Patient | | Treatment | Treatment | Duration of | |
|---|---|---|---|---|---|
| no. | Sex/age | location | form | treatment | Treatment result |
| S1¹⁾ | man/68 | Right foot | cyanoacrylate | about 3 times daily, | distinct healing after 1 |
| | | | application | additional daily | week's treatment, skin |
| | | | | treatments as | normalized, still symptom |
| | | | | required at e.g. | free without fissures after |
| | | | | personal hygiene or | 6 months on sustained |
| | | | | pain | filing off of callous skin |
| S2²⁾ | woman/75 | Left foot 1^{st} | skin removal | as required | no visible result after 1 |
| | | treatment | by filing off | | week |
| | | | skin callosity | | |
| | | | followed by | | |
| | | | bandaging | | |
| S3²⁾ | man/83 | Left foot 2^{nd} | cyanoacrylate | as right foot | as right foot |
| | | treatment | application | | |
| | | (following 1^{st} | | | |
| | | treatment) | | | |
| S4²⁾ | man/78 | Right foot | cyanoacrylate | about 3 times within | Removal of skin |
| | | | application | 24 hours | corresponding to an |
| | | | | | extent allowing painless |
| | | | | | cleansing of fissure. |
| | | | | | Subsequently quick |
| | | | | | healing |

¹⁾Type 2 diabetes and severe callosity of skin on both feet especially on pads; in addition fissures and light haemorrhages.
²⁾Diabetes and severe callosity of skin on both feet especially on pads; in addition fissures and light haemorrhages.
³⁾Diabetes. Deeply infected fissure on lateral part of right foot. The foot had previously been treated by medical specialist with antibiotics per os and locally but without effect. The depth of the fissure did not allow for cleaning out the infection without great pain.

Four patients (S 1-S4) had skin removed from wounds requiring treatment on one foot

Patients S1 and S3 displayed all identical positive treatment response in relation to dermatological treatment, and the results are therefore jointly given in Table II above.

Patients S1 and S4 had severe pains when the pads were loaded during walking. The pains disappeared after healing. A simple method for skin removal and cleaning out of fissures is given here. The method is simple and painless and has a convincing effect as shown in Table II. It is to be noted that the sooner callous skin is removed, the smaller the risk of fissures being generated, of a possible infection arise in the fissures, and of this infection possibly spreading.

### Example 9

### Comparative Study of Patients with Prurigo Besnier

Ten patients (PB1-PB10) with Prurigo Besnier were part of the test. Areas with eczema localized to skin folds on arms and/or legs were treated with cyanoacrylate application and conventional treatment in form of Elocon® cream respectively (Group 3 steroid) in fold of right and left extremity respectively. Each patient thus served as his own comparative control.

The results are given in Table III below, of which the dramatic and effective effect of cyanoacrylate application appears with convincing clearness.

TABLE III

| Patient | Treatment | Treatment | Duration of | | After-treatment/ |
|---|---|---|---|---|---|
| No. | location | form | treatment | Treatment result | final result |
| PB1-PB5 | Right arm skin | Elocon ® | 1 to 2 times a | Some improvement | Elocon ® cream |
| | fold | cream | day for about 7 | | for not visible |
| | | | days | | eczema, about 1 |
| | | | | | week |
| | Left arm skin | Cyanoacrylate | 1 time over the | After removal of the | Group 1 steroid |
| | fold | application | skin area | solid polymer coat, | until 1-2 days/no |
| | | | requiring | the treated skin area | visible eczema |
| | | | treatment | appeared intact with | |
| | | | | microscopic, spot | |
| | | | | haemorrhages (where | |
| | | | | cyanoacrylate had | |
| | | | | penetrated down into | |
| | | | | subcutis) | |
| PB6-PB10 | Right leg skin | Elocon ® | 1 to 2 times a | Some improvement | Elocon ® cream, |
| | fold | cream | day for about 7 | after about 7 days | until not visible |
| | | | days | | eczema, about 1 |
| | | | | | week |
| | Left leg skin | Cyanoacrylate | 1 time over the | After removal of the | Group 1 steroid |
| | fold | application | skin area | solid polymer coat, | for 1-2 days/no |
| | | | requiring | the treated skin area | visible eczema |
| | | | treatment | appeared intact with | |
| | | | | microscopic, spot | |
| | | | | haemorrhages (where | |
| | | | | cyanoacrylate had | |
| | | | | penetrated down into | |
| | | | | subcutis) | |

Summarized it can be concluded that the conventional steroid treatment can be reduced to a hitherto unknown minimum. An often long-term treatment with group III steroids can now be replaced by a cyanoacrylate application supplemented with a short treatment with group I steroids.

### Example 10

### Comparative Study of Patients with Psoriasis Plaque

Six patients (P1-P6) with Psoriasis plaques. Areas localized to skin folds on arms and/or legs were treated. Left skin fold was treated with conventional treatment with group 1-4 steroid creams, and right skin fold was pretreated with cyanoacrylate application and after-treated with group 1-3 steroid cream. Each patient thus served as his own comparative control.

Summarized it can be concluded that the conventional steroid treatment can be reduced substantially. The often long-term treatment with high-group steroids can now be replaced by a cyanoacrylate application supplemented with a treatment with a low-group steroid for a short time.

TABLE IV

| Patient | Treatment | Treatment | Duration of | | After-treatment/ |
|---|---|---|---|---|---|
| No. | location | form | treatment | Treatment result | final result |
| P1 | Right arm | Group 3 | 1 to 2 times a | Some improvement | None/some |
| | skin fold | steroid | day for about | | improvement but |
| | | | two weeks | | still visible plaques |
| | Left arm skin | Cyanoacrylate | 1 time over the | Loose plaques were | Group 1 steroid for |
| | fold | application | skin area | caught and encapsulated | about 4 days. After |
| | | | requiring | in the solid polymer coat. | that no visible |
| | | | treatment | The treated skin area | loose plaques |
| | | | | appeared intact with a few | |
| | | | | areas with microscopic | |
| | | | | spot haemorrhages | |
| P2 | Right arm | Group 4 | 1 to 2 times a | Some improvement | Changed to |
| | skin fold | steroid | day for about | | cyanoacrylate |
| | | | two weeks | | treatment like the |
| | | | | | left arm skin fold |
| | Left arm skin | Cyanoacrylate | like P1 | like P1 | Group 3 steroid for |
| | fold | application | | | about 6 days. After |
| | | | | | that, no visible |
| | | | | | loose plaques |
| P3¹⁾ | Right arm | Group 4 | 3 times a day | Some improvement | Changed to |
| | skin fold | steroid | for about two | | cyanoacrylate |
| | | | weeks | | treatment like the |
| | | | | | left arm skin fold |
| | Left arm skin | Cyanoacrylate | Like P1 | Like P1 | Group 3 steroid for |
| | fold | application | | | about 6 days. After |
| | | | | | that, no visible |
| | | | | | loose plaques |
| P4 | Right leg | Group 3 | 1 to 2 times a | Good improvement | None |
| | skin fold | steroid | day for about 7 | | |
| | Left leg skin | Cyanoacrylate | like P1 | Like P1 | Group 1 steroid for |
| | fold | application | | | about 2 days. After |
| | | | | | that, no symptoms |
| P5 | Right leg | Group 4 | 3 times a day | Good improvement | Recurrence after 2 |
| | skin fold | steroid | for about two | | weeks |
| | | | weeks | | |
| | Left leg skin | Cyanoacrylate | like P1 | like P1 | Group 3 steroid for |
| | fold | application | | | about 4 days. After |
| | | | | | that, no visible loose |
| | | | | | plaques, no |
| | | | | | recurrence |
| | | | | | observed within 6 |
| | | | | | weeks |
| P6¹⁾ | Right leg | Group 4 | 1 to 2 times a | Slight improvement but still | None. Changed to |
| | skin fold | steroid | day for about | many loose plaques | cyanoacrylate |
| | | | two weeks | | treatment like left |
| | | | | | arm skin fold |
| | Left leg skin | Cyanoacrylate | 2 times of the | Loose plaques only partly | Group 3 steroid for |
| | fold | application | skin area | caught and encapsulated | about 6 days. After |
| | | | requiring | in the first polymer coat. | that, no visible |
| | | | treatment | After 2 treatments like P1, | loose plaques, no |
| | | | | with microscopic spot | recurrence |
| | | | | haemorrhages | observed with 6 |
| | | | | | weeks |

¹⁾Severe plaque formations

### Example 11

### Initial Test: Removal of Crust from Small Superficial Wounds

Wounds of six patients with wounds of 1-2 cm×1-2 cm were painted with cyanoacrylate. The crust was removed after polymerization easily and with no pain in all patients. No larger haemorrhages observed but a few and sparse between 0.5 mm×0.5 mm, spot small haemorrhages. Two of the patients had additional wounds which were removed surgically for comparison. Pain and larger haemorrhages observed.

### Example 12

### Revision of Superficial Wounds on Crust

Seven patients (R1-R7) with aetiologically different, not or only a little infected wounds on crust were treated with cyanoacrylate without prior anaesthesia. The results are given in Table V below.

All patients had previously had similar wounds, for which they had been treated with surgical removal of crust by means of a scalpel and tweezers during preceding medication with Diazepam® per os and Ketogan®.

TABLE V

| | | Wound size/ | | |
|---|---|---|---|---|
| | | duration | | |
| | | before | | |
| Patient | | cyanoacrylate | | After-treatment/Treatment |
| No. | Aetiology | treatment | Treatment | result |
| R1¹⁾ | Cardio- | 5 cm × 6 cm, | Previously tried: | 1 add. cyanoacry-late application |
| ♀, | vascular | slight infection/ | Fucidin compress, anti-biotics | to remove necrotic tissue/ |
| 78 years | disease | about 6 | per os. Wound removal with | Frequent bandage change. |
| | oedema | months | scalpel not possible due to | Wound completely healed after |
| | | | pain. | three weeks |
| | | | Alternatively thereafter: | |
| | | | 1 cyanoacrylate application, | |
| | | | pulling off of crust. Cleansing | |
| R2¹⁾ | Cardio- | 5 cm × 8 cm/ for | Previously tried: | Like R1 |
| ♂, | vascular | about 6 | Fucidin ointment and | |
| 82 years | disease | months | bandaging | |
| | | | Alternatively thereafter: | |
| | | | 1 cyanoacrylate application, | |
| | | | pulling off of crust. Cleansing | |
| R3¹⁾ | Cardio | about 5 cm in | Like R2 | Like R1 |
| ♀, | vascular | diameter/ | | |
| 84 years | disease | about 8 | | |
| | | months | | |
| R4¹⁾ | Type II | 3-5 × cm × 3-5 cm/ | Like R2 | Frequent bandage changes. After |
| ♀, | diabetes | about 12 | | one week, distinct granulation. |
| 76 years | over- | months | | Total healing after 6 weeks |
| | weight | | | |
| R5¹⁾ | Type II | 3-5 × cm × 3-5 cm/ | Like R2 | Frequent bandage changes. After |
| ♀, | diabetes | about 12 | | one week, distinct granulation. |
| 84 years | over- | months | | Total healing after 6 weeks |
| | weight | | | |
| R6¹⁾ | Type I | 3-5 × cm × 3-5 cm/ | Like R2 | Frequent bandage changes. After |
| ♂, | diabetes, | about 12 | | one week, distinct granulation. |
| 72 years | over- | months | | Total healing after 8 weeks |
| | weight | | | |
| R7¹⁾ | Type I | 3-5 × cm × 3-5 cm/ | Like R2 | Frequent bandage changes. After |
| ♂, | diabetes, | about 12 | | one week, distinct granulation. |
| 80 years | over- | months | | Total healing after 7 weeks |
| | weight | | | |
| R8 | Large | Left: | Previously: | Careful personal hygiene just like |
| ♀, | mammae | irritation and | None | left mammae. Slow healing |
| 65 years | | humidity | Thereafter: | |
| | | | None | |
| | | Right: | Previously: | Careful personal hygiene, healing |
| | | Suppurating | None | began quickly to complete healing |
| | | wounds | Thereafter: | after 1 month |
| | | | cyanoacrylate application 3 | |
| | | | times a day for 3 days | |

¹⁾After cyanoacrylate treatment and the subsequent removal of crust, no bandage cont. medicine, such as antibiotics, chlorhexidine or hydrogen peroxide, were used. Wounds only kept clean with sterile saline water and frequent bandage changes.

What is claimed is:

1. A method for treating exanthema on dermal or mucous membrane tissues, the method comprising:
applying a sufficient amount of medicament onto a selected area of tissue, the medicament including a cyanoacrylate; and
allowing the cyanoacrylate to polymerize and form a coat adhered to the selected area of tissue;
wherein the cyanoacrylate has the general formula I: 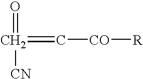
wherein R is chosen from the group of alkyls or alkenyls having 1 to 10 carbon atoms, cycloalkyls having 5 to 10 carbon atoms, phenyl, 2-ethoxyethyl, 3-methoxybutyl, arenes or alkyl-substituted arenes, or a substituent having the formula II: 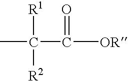
wherein R¹ and R² are chosen independently of each other from the group consisting of hydrogen, ethyl, propyl or butyl, and R″ is chosen from the group consisting of alkyls or alkenyls having 1 to 10 carbon atoms, cycloalkyls having 3 to 10 carbon atoms, phenyl, benzyl, methylbenzyl, phenylbenzyl, halogen-substituted or alkyl-substituted compounds thereof.

2. The method of claim 1, further including removing exanthema-plaque from the selected area of the tissue by removing the cyanoacrylate coat, which is adhered to the plaque, away from the tissue.

3. The method of claim 1, wherein the exanthema is selected from a group consisting of atopic exanthema, seborrhoeic exanthema, discoid exanthema, allergic contact dermatitis, irritation contact dermatitis, and psoriasis exanthema.

4. The method of claim 1, wherein the exanthema is a fungal infection caused by Trichophyton, Epidermophyton, Candida, Torulosis, Cryptococcus, Pityrosporon, Trochosporon, Candida albicans or Pityriasis versicolor.

5. The method of claim 1, wherein the medicament includes a combination of cyanoacrylates, wherein at least one of the combination of cyanoacrylates has a different R group.

6. The method of claim 5, wherein the at least one cyanoacrylate has ethyl as the R group

7. The method of claim 1, wherein the medicament further includes at least one additive selected from the group consisting of a stabilizer to prevent the medicament from spontaneously polymerizing during storage, an agent to accelerate a polymerization reaction and colorant.

8. The method of claim 7, wherein the stabilizer of the medicament has a pH equal to or under 7 and the medicament is neutralized upon contact with moisture.

9. The method of claim 7, wherein the agent to accelerate the polymerization reaction is a C1-C10 alkane, a ketone or alcohol.

10. The method of claim 1, wherein the medicament is the form of a liquid or a gel.

11. A method of treating a viral infection on dermal tissue or mucous membrane tissue, the method comprising:
applying medicament onto an infected area of the tissue, the medicament including more than one cyanoacrylate, and an additive;
allowing the cyanoacrylate to polymerize and form a coating on the infected area of tissue such that further development of the viral infection is inhibited, wherein the more than one cyanoacrylate has the general formula I: 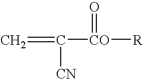
wherein R is chosen from the group of alkyls or alkenyls having 1 to 10 carbon atoms, cycloalkyls having 5 to 10 carbon atoms, phenyl, 2-ethoxyethyl, 3-methoxybutyl, arenes or alkyl-substituted arenes, or a substituent having the formula II: 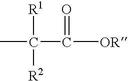
wherein R¹ and R² are chosen independently of each other from the group consisting of hydrogen, ethyl, propyl or butyl, and R″ is chosen from the group consisting of alkyls or alkenyls having 1 to 10 carbon atoms, cycloalkyls having 3 to 10 carbon atoms, phenyl, benzyl, methylbenzyl, phenylbenzyl, halogen-substituted or alkyl-substituted compounds thereof.

12. The method of claim 11, wherein the viral infection is caused by herpes simplex 1, herpes simples 2, herpes zoster or papilloma virus.

13. A method for revising a wound located on dermal or mucous membrane tissue, the method comprising:
applying a sufficient amount of a cyanoacrylate to penetrate into cavities of the a crust of the wound;
allowing the cyanoacrylate to polymerize and adhere to the crust;
removing the crust adhered to the polymerized cyanoacrylate by pulling the cyanoacrylate from the dermal or mucous membrane tissue.

14. The method of claim 13, wherein the method further includes cleaning an exposed, open lesion created by the removal of the wound crust.

15. The method of claim 14, wherein the method further includes applying a medicament on the open lesion created by the removal of the wound crust.

16. The method of claim 15, wherein the medicament is selected from the group consisting of an antibiotic, a fungicidal drug, antibacterial, and an eczema-treating drug.

17. The method of claim 13, wherein the crust includes necrotic tissue.

18. A method for removing at least a portion of a wart or condyloma from dermal tissue, the method comprising:
applying a sufficient amount of a cyanoacrylate to at least partially encapsulate the wart or condyloma;
allowing the cyanoacrylate to polymerize;
removing at least a portion of the wart or condyloma which is at least partially encapsulated within the polymerized cyanoacrylate by pulling the cyanoacrylate from the dermal tissue.

19. The method of claim 18, wherein an excess of cyanoacrylate is applied to the wart or condyloma such that a flap for gripping is defined by the cyanoacrylate.

20. A method for maintaining in close proximity separated tissue edges of a wound site during a tissue closure procedure, the method comprising:
applying a sufficient amount of cyanoacrylate at the separated edges of a wound site such that gripping flaps are formed;
allowing the cyanoacrylate to cure; and
grasping the gripping flaps to place the edges of the wound in close proximity to facilitate the tissue closure procedure.
